(19) United States
(12) Patent Application Publication
(10) Pub. No.: US 2021/0402034 A1
(43) Pub. Date: Dec. 30, 2021

(54) FLUORORUBBER WAX-MELTING TRAY AND CLEANING METHOD THEREOF

(71) Applicant: SHENZHEN XIAOYUAN IMPORT & EXPORT LTD, SHENZHEN (CN)

(72) Inventors: Xiaoyuan MA, Shenzhen (CN); ALAN KENT FARRELL, SHENZHEN (CN)

(21) Appl. No.: 17/211,797

(22) Filed: Mar. 24, 2021

(30) Foreign Application Priority Data
Jun. 30, 2020 (CN) 2020106093819

(51) Int. Cl. A61L 9/03; C08L 19/00; B08B 9/08; B08B 7/02

(52) U.S. Cl. CPC A61L 9/03; B08B 7/02; B08B 9/08; C08L 19/00

(57) ABSTRACT

A fluororubber wax-melting tray and a cleaning method thereof are disclosed. The fluororubber material of the disclosure is a synthetic polymer elastomer with high temperature resistance, corrosion resistance and low wear resistance. The fluororubber wax-melting tray has the same wax melting effect as the existing ceramic or metal wax melting trays. The shape of the wax melting tray is changed by the external force exerted by pushing the bottom of the fluororubber wax melting tray lightly. When both the external force tending to change the shape of the wax-melting tray and the adsorption force between the wax molecules are greater than the adsorption forces between the fragrance wax and the wax melting tray, the absorption force between a wax piece and the wax melting tray is released, and the whole solidified wax piece can be completely peeled off.

### CROSS-REFERENCE TO RELATED APPLICATIONS

The present application claims foreign priority of Chinese Patent Application No. 202010609381.9, filed on Jun. 30, 2020 in the National Intellectual Property Administration, the disclosures of all of which are hereby incorporated by reference.

### TECHNICAL FIELD

The disclosure relates to the technical field of wax-melting trays, and more specifically, to a fluororubber wax-melting tray and a cleaning method thereof.

### BACKGROUND

Wax melting tray, mainly being placed on top of all kinds of aromatherapy furnace for melting the wax pieces of all kinds of fragrance, has been widely used in household health care, health conditioning, odor removal. In response for the call of China by improving the quality of people's lives, the environment and preventing diseases, the wax melting tray has been produced. Tradition of home aromatherapy originated in Europe and the United States, and the penetration rate of families applying aromatherapy furnaces in European and American countries is very high. With the continuous improvement of the living standard of the people in China, requirements for the quality of life are also constantly improving. In recent years, aromatherapy has become popular in China. Traditional wax-melting trays are mainly made of ceramics and metals, and the high thermal conductivity of the two materials leads to frequent high-temperature scald. And a more serious problem is that the trays made of these two materials are not easy to clean after use, which reduces the service life of the trays. The conventional wax tray, which is a solid without elasticity, after the wax is melted at a high temperature and is solidified again, an adsorption force is generated between the wax and the wax melting tray. When a clean up of the wax is required, other sharp tools, such as a blade, must be used to cut down the remaining wax bit by bit. This method is neither safe nor sanitary, and also time consuming. It may take a few minutes to clean up.

### SUMMARY

In view of the above, the present disclosure provides a fluororubber wax melting tray and a cleaning method thereof. The fluororubber wax melting tray adopt a specific material and effectively solve the problems of the prior art. The present disclosure is realized by the following technical scheme.

In a first aspect, the disclosure provides a fluororubber wax-melting tray, which includes a fluororubber wax-melting tray body for melting various fragrant wax pieces placed on various vaporizers.

Further, the fluororubber wax-melting tray is made of a fluororubber material. The fluororubber material includes an FKM gum, a filler, an active agent and a processing aid as ingredients.

In a second aspect, the disclosure provides a cleaning method of the fluororubber wax-melting tray, including pushing a bottom portion of the fluororubber wax melting tray; releasing an adsorption force between the aroma wax and the wax-melting tray body when an external force tend for changing the shape of the fluororubber wax melting tray and the adsorption force between the fragrance wax molecule are larger than the adsorption force between the aroma wax and the fluororubber wax-melting tray; an entire set of the aroma wax is peeled off.

Further, after cleaning, the fluororubber wax-melting tray can be reused.

In the fluororubber wax-melting tray provided by the disclosure, the fluororubber material is adopted, so that the problem of high-temperature scalding cause by high thermal conductivity of ceramics and metals is avoided. More importantly, it completely changes the cleaning problem after the use of the fragrant wax. By changing the shape of the wax-melting tray with external force, the adsorption force between the wax pieces and the wax melting tray is released, and the whole solidified wax piece can be completely peeled off, thus greatly saving the time for cleaning the wax tray.

### BRIEF DESCRIPTION OF THE DRAWINGS

FIG. 1 is a schematic diagram of the fluororubber wax-melting tray of the disclosure;

FIG. 2 is a sectional view of the fluororubber wax-melting tray of the disclosure.

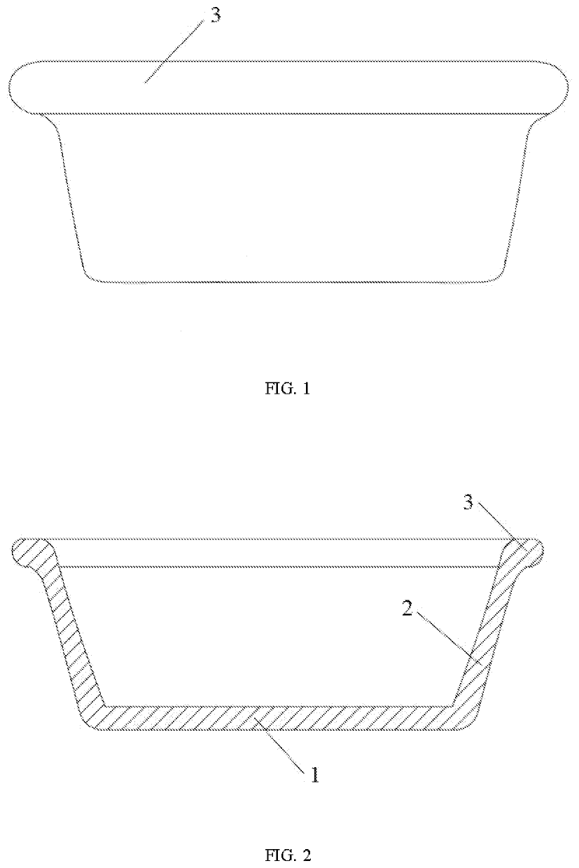

In the drawings:
1, bottom of the fluororubber wax-melting tray;
2, fluororubber wax-melting tray side wall;
3, upper edge of the fluororubber wax melting tray.

### DETAILED DESCRIPTION OF EMBODIMENTS

Hereinafter, embodiment of the present disclosure will be described in detail with reference to the accompany drawings.

The embodiments of the present disclosure are illustrated below by specific examples, and other advantages and functions of the disclosure will be readily apparent to those skilled in the art from the disclosure herein. It will be apparent that the described embodiments are only a portion of the embodiments of the present disclosure and not the entire embodiment. The present disclosure may also be practiced or applied by other different embodiments, and the details in this specification may be based on different viewpoints and applications. Various modifications or changes may be made without departing from the spirit of the present disclosure. It should be noted that the following embodiments and the features in the embodiments may be combined with each other without conflict. Based on the embodiments in this disclosure, all other embodiments obtained by those of ordinary skill in the art without making creative efforts are within the scope of this disclosure.

The disclosure discloses a fluororubber wax-melting tray, which adopts a specific material and effectively solves the problems of the prior art. Specific examples are as follows:

The disclosure relates to a fluororubber wax-melting tray, which includes a fluororubber wax-melting tray body, and is being placed on a plurality of vaporizers to melt wax pieces with various scents. The appearance, shape and size of the fluororubber wax-melting trays are not limited. Other auxiliary parts can be included, and the connection relationship with the fluororubber wax-melting tray is not limited.

The fluororubber wax-melting tray is made of the fluororubber material, and the ingredients of the fluororubber materials include a FKM raw rubber, a filler, an active agent and a processing aid. Preferably, the proportion by weight of ingredients is respectively 74.51% for FKM gum, 17.14% for filler, 6.71% for active agent and 1.64% for processing aid. The weight proportion of the components may also be changed according to actual needs, and the change in weight proportion shall not be taken as a limitation to the present disclosure.

The traditional wax melting tray is a non-elastic solid, which is mainly made of ceramics and metals. After the fragrant wax is melted at high temperature and solidified again, it will have a firm adsorption effect with the wax melting tray. If the wax is to be cleaned, other sharp tools, such as a blade, need to be used for removing the attached wax bit by bit. This approach is neither safe nor sanitary, and time consuming. After detailed studies of the characteristics of fragrance wax, a fact is found that although fragrance wax has adsorption to all materials, but the degree of adsorption would be different. The experimental results show that the adsorption of fragrance wax itself is much greater than the adsorption between fragrance wax and wax melting tray. So the idea is that if elasticity is added to the physical properties of the wax tray and a elastic wax tray come out, then the shape of the wax tray could be changed by applying an external force. When the external force tending to change the shape of the wax melting tray and the adsorption force between the fragrance wax molecules are greater than the adsorption forces between the wax and the wax melting tray, the adsorption power between the wax piece and the wax melting tray is released, and the whole wax piece falls off. Ordinary rubber density coefficient is smaller. Under high temperature, the molecular structure will be deformed, the gap will become large, so there will be leakage defects. After hundreds of experiments, the fluororubber can achieve the desired effect perfectly is concluded.

The fluororubber is a synthetic polymer elastomer in which a fluorine atom is bonded to a carbon atom in a main chain or a side chain of its molecular structure. The fluorine atoms provide the rubber with excellent heat resistance, oxidation resistance, oil resistance, corrosion resistance and wear resistance. This kind of material can solve the problem that ordinary rubber seeps wax at high temperature.

Based on the properties of wax piece after solidification and the elasticity coefficient of fluororubber, through hundreds of tests, the wax melting tray was found. The utility model not only avoids the problem of high-temperature scald caused by high thermal conductivity of ceramics and metals, but also completely changes the cleaning problem after the wax is used.

The cleaning method of the fluororubber wax-melting tray is as follows: gently pushing the bottom of the melting tray, external force changes the shape of the wax melting tray, and the adsorption force between the wax piece and the wax melting tray is released, so that the whole solidified wax piece can fall off.

When the magnitude of external force tending to change the shape of the fluoro-rubber wax melting tray is less than the adsorption force between the fragrance wax molecule and greater than the absorption force between fragrance wax and the fluoro rubber-melting wax tray, the adsorption power between the wax piece and the wax tray is released, and the whole fragrance wax falls off.

In practice, the fluororubber wax-melting tray can be used repeatedly.

The above is only to illustrate the embodiments of the present disclosure, and is not limited to the present disclosure. For those skilled in the art, within the spirit and principles of the disclosure, Any modification, equivalent substitution, improvement and the like without going through creative work shall be included in the scope of protection of the present disclosure.

What is claimed is:

1. A fluororubber wax-melting tray, comprising a wax-melting tray body; the wax-melting tray body is being placed on a plurality of vaporizers for melting wax with various scents.

2. The fluororubber wax-melting tray of claim 1, wherein the fluororubber wax-melting tray is made of a fluororubber material.

3. The fluororubber wax-melting tray of claim 2, wherein the fluororubber material comprises an FKM gum, a filler, an active agent and a processing aid as ingredients.

4. A method for cleaning the fluororubber wax-melting tray of claim 1, comprising
pushing a bottom portion of the fluororubber wax melting tray;
releasing an adsorption force between the aroma wax and the wax-melting tray body when an external force tend for changing the shape of the fluororubber wax melting tray and the adsorption force between the fragrance wax molecule are larger than the adsorption force between the aroma wax and the fluororubber wax-melting tray; an entire set of the aroma wax is peeled off.

5. The method of claim 4, wherein the cleaned fluororubber wax-melting tray can be used repeatedly.
